(19) United States
(12) Patent Application Publication
(10) Pub. No.: US 2025/0352363 A1
(43) Pub. Date: Nov. 20, 2025

(54) ORTHOPEDIC TRIAL SYSTEMS

(71) Applicant: Zimmer, Inc., Warsaw, IN (US)

(72) Inventor: Andrew Scott Payton, Warsaw, IN (US)

(21) Appl. No.: 19/208,250

(22) Filed: May 14, 2025

Related U.S. Application Data
(60) Provisional application No. 63/649,029, filed on May 17, 2024.

(51) Int. Cl. A61F 2/46; A61F 2/30; A61F 2/40

(52) U.S. Cl. CPC A61F 2/4684; A61F 2/40; A61F 2002/30604

(57) ABSTRACT

Various systems, methods and assemblies are described including a modular trial assembly for an arthroplasty. The modular trial assembly can include: a trial tray having an aperture therein; a trial bearing coupled to the trial tray and configured to be selectively moveable relative thereto to adjust a height of an articular surface of the trial bearing relative to the trial tray; a connector received by the aperture and extending from the trial tray; and a post coupled to the connector and positioned adjacent the trial tray, wherein the post is configured to be received by an orthopedic prosthesis for coupling the modular trial assembly to the orthopedic prosthesis.

### CLAIM OF PRIORITY

This application claims the benefit of U.S. Provisional Patent Application Ser. No. 63/649,029, filed on May 17, 2024, the benefit of priority of which is claimed hereby, and which is incorporated by reference herein in its entirety.

### FIELD OF THE DISCLOSURE

The present disclosure relates to an orthopedic prostheses that can be used for trialing during an arthroplasty procedure such as a reverse shoulder or another total or partial joint replacement.

### BACKGROUND

In the human body, tissue can require repair and replacement. Such tissue includes bone, muscles, tendons, ligaments and cartilage. For example, disease can necessitate replacement of bone(s) of the joint with one or more prosthetic components. Such replacement can require use of orthopedic guides, trialing systems and other instruments to facilitate proper orientation and/or size of the one or more prosthetic components.

The human shoulder joint may require repair or replacement. A conventional or reverse joint replacement may be used in a situation where the bone is diseased and/or a rotator cuff is damaged or lacking. This can provide pain relief and return the shoulder joint to normal kinematic function (e.g., the patient can again raise their arm above their head).

### SUMMARY

The present disclosure provides trial orthopedic prostheses including trial humeral trays, trial bearings and other components. The present disclosure provides systems that include such trial prostheses, which can be used in a shoulder arthroplasty. Components such as the trial bearing are shown configured for a reverse shoulder arthroplasty. Additionally, the concepts discussed herein can be extended to other trial prostheses and to other joints of the human body, and thus, can be applicable to orthopedic prostheses and instruments for the hip, knee, ankle, etc. It should be noted that the humeral trial trays and bearing trials discussed herein may otherwise be known as an instrument and are not implanted permanently within a patient's anatomy. Rather, one of the humeral trial trays and one of bearing trials are temporarily attached to a humeral prosthesis or the trial humeral implant and are placed in the shoulder joint to simulate a permanent humeral tray and permanent bearing to check for appropriate joint kinematics such as range of motion, etc.

The present inventor has realized trial systems have a proliferation of variants. For example, humeral trial tray systems typically come with a plurality of different implants each having a different stock size that simulates a particular permanent tray stock size and each having a different offset that simulates a particular offset for the permanent tray. Thus, it is not atypical for humeral trial tray systems to include ten or more humeral trial tray components. Additionally, trial bearing systems typically come in a plurality of different sizes and in a plurality of different shapes. Thus, the combination of such humeral trial tray and trial bearing systems have a large number of components. The present inventor has recognized that the number of components for such trial systems (both humeral trial tray systems and bearing trial systems) can be reduced and cost can be saved.

Additionally, the present inventor has recognized that the humeral trial trays discussed herein can be made compatible with various humeral prostheses or trial humeral implant designs. Surgeons may have a particular preference for a particular prosthesis system due to the patient's anatomy, surgical approach preference, or familiarity with the product and the surgical technique employed with the particular system. Humeral prostheses for a reverse shoulder arthroplasty may be single piece stem components or can have a stemless construction. The present inventor has recognized humeral trial systems that include posts of various constructions (e.g., different sizes and shapes including lengths). These plurality of posts can allow the humeral trial trays to be connected to various different designs for the humeral prosthesis or trial humeral implant. Thus, system compatibility is increased.

Furthermore, the present inventor has recognized the trial systems disclosed herein can be coupled to the humeral prosthesis or trial humeral implant and the humeral trial tray can adjusted to a desired offset and articular surface height position in situ. This adjustability in situ can save time and reduce surgical complexity.

The above discussion is intended to provide an overview of subject matter of the present patent application. It is not intended to provide an exclusive or exhaustive explanation of the invention. The description below is included to provide further information about the present patent application.

To better illustrate the orthopedic trial apparatuses and orthopedic trialing systems disclosed herein, a non-limiting list of examples is provided here:

Example 1 is a modular trial assembly for an arthroplasty optionally comprising: a trial tray having an aperture therein; a trial bearing coupled to the trial tray and configured to be selectively moveable relative thereto to adjust a height of an articular surface of the trial bearing relative to the trial tray; a connector received by the aperture and extending from the trial tray; and a post coupled to the connector and positioned adjacent the trial tray, wherein the post is configured to be received by an orthopedic prosthesis for coupling the modular trial assembly to the orthopedic prosthesis.

In Example 2, the subject matter of Example 1 optionally includes, wherein the aperture is a slot and the connector is selectively moveable along the slot to adjust a position of the post relative to the trial tray.

In Example 3, the subject matter of Example 2 optionally includes, wherein the trial bearing includes a slot therein, wherein the slot of the trial bearing allows for access to the connector and the slot of the trial tray.

In Example 4, the subject matter of Examples 1-3 optionally includes, wherein the trial tray includes a plurality of tabs, wherein the plurality of tabs are spaced apart at a desired distance, and wherein one or more of the plurality of tabs are configured to be received in one or more of a plurality of grooves of the trial bearing.

In Example 5, the subject matter of Example 4 optionally includes, wherein the trial tray and the trial bearing are selectively coupled via the one or more of the plurality of tabs being received by the one or more of the plurality of grooves, and wherein the trial bearing is selectively rotatable relative to the trial tray to remove the one or more of the plurality of tabs from the one or more of the plurality of grooves to allow for selective movement of the trial bearing relative to the trial tray to adjust the height of the articular surface of the trial bearing.

In Example 6, the subject matter of Example 5 optionally includes, wherein the trial bearing is selectively rotatable to align the plurality of tabs with at least one window of the trial bearing to allow for selective movement of the trial bearing relative to the trial tray to adjust the height of the articular surface of the trial bearing.

Example 7 is an orthopedic system for an arthroplasty optionally comprising: a trial tray; a trial bearing configured to be selectively coupled to and decoupled from the trial tray by rotation of the trial bearing relative to the trial tray, wherein, when decoupled from the trial tray, the trial bearing is selectively moveable to adjust a height of an articular surface of the trial bearing relative to the trial tray; a post configured to be received by an orthopedic prosthesis; and a connector configured to couple the post with the trial tray.

In Example 8, the subject matter of Example 7 optionally includes, wherein, when the connector is coupled to the trial tray and the post, the trial tray includes a slot and the connector is configured to be selectively movable along the slot adjust a position of the post relative to the trial tray.

In Example 9, the subject matter of Example 8 optionally includes, the trial bearing includes a slot therein, wherein, when the trial bearing is coupled to the trial tray, the slot of the trial bearing allows for access to the connector and the slot of the trial tray.

In Example 10, the subject matter of Examples 7-9 optionally includes, wherein the trial tray includes a plurality of tabs, wherein the plurality of tabs are spaced apart at a desired distance, and wherein when the trial bearing and the trial tray are coupled, one or more of the plurality of tabs are configured to be received in one or more of a plurality of grooves of the trial bearing, and wherein the trial bearing is selectively rotatable relative to the trial tray to remove the one or more of the plurality of tabs from the one or more of the plurality of grooves to allow for selective movement of the trial bearing relative to the trial tray to adjust the height of the articular surface of the trial bearing.

In Example 11, the subject matter of Example 10 optionally includes, wherein the trial bearing is selectively rotatable to align the plurality of tabs with at least one window of the trial bearing to allow for selective movement of the trial bearing relative to the trial tray to adjust the height of the articular surface of the trial bearing.

Example 12 is a modular trial assembly for an arthroplasty optionally comprising: a trial tray having an aperture therein; a trial bearing coupled to the trial tray and configured to be rotatable and selectively linearly translatable relative to the trial tray to adjust a height of an articular surface of the trial bearing relative to the trial tray; and a post coupled to the trial tray and connecting the modular trial assembly to an orthopedic prosthesis.

In Example 13, the subject matter of Example 12 optionally includes, a connector connecting the post to the trial tray, wherein the connector is received in a slot of the trial tray, and wherein the connector is selectively moveable along the slot to adjust a position of the post relative to the trial tray.

In Example 14, the subject matter of Example 13 optionally includes, wherein the trial bearing includes a slot therein, wherein the slot of the trial bearing allows for access to the connector and the slot of the trial tray.

In Example 15, the subject matter of Examples 12-14 optionally includes, wherein the trial tray includes a plurality of tabs, wherein the plurality of tabs are spaced apart at a desired distance, and wherein one or more of the plurality of tabs are configured to be received in one or more of a plurality of grooves of the trial bearing, wherein the trial tray and the trial bearing are selectively coupled via the one or more of the plurality of tabs being received by the one or more of the plurality of grooves, and wherein the trial bearing is selectively rotatable relative to the trial tray to remove the one or more of the plurality of tabs from the one or more of the plurality of grooves to allow for selective movement of the trial bearing relative to the trial tray to adjust the height of the articular surface of the trial bearing.

In Example 16, the subject matter of Example 15 optionally includes, wherein the trial bearing is selectively rotatable to align the plurality of tabs with at least one window of the trial bearing to allow for selective movement of the trial bearing relative to the trial tray to adjust the height of the articular surface of the trial bearing.

Example 17 is a method of trialing for an arthroplasty optionally comprising: providing a trial tray and a trial bearing; aligning the trial bearing with the trial tray in a first orientation; inserting the trial bearing down onto the trial tray in the first orientation; and rotating the trial bearing relative to the trial tray from the first orientation to a second orientation to couple the trial bearing to the trial tray.

In Example 18, the subject matter of Example 17 optionally includes, rotating the trial bearing relative to the trial tray from the second orientation back to the first orientation; and in the first orientation, linearly translating the trial bearing relative to the trial tray to adjust a height of an articular surface of the trial bearing relative to the trial tray.

In Example 19, the subject matter of Examples 17-18 optionally includes, wherein aligning the trial bearing with the trial tray includes aligning a plurality of tabs of the trial tray with at least one window of the trial bearing, and wherein rotating the trial bearing relative to the trial tray from the first orientation to a second orientation inserts one or more of the plurality of tabs into one or more of a plurality of grooves of the trial bearing.

In Example 20, the subject matter of Examples 17-19 optionally includes, connecting a post to the trial tray using a connector inserted in a slot of the trial tray; and adjusting a position of the post relative to the trial tray by moving the connector along the slot.

In Example 21, the subject matter of Example 20 optionally includes, accessing the connector for movement in the slot of the trial tray via an aperture in trial bearing.

Example 22 is at least one machine-readable medium including instructions that, when executed by processing circuitry, cause the processing circuitry to perform operations to implement of any of Examples 1-21.

Example 23 is an apparatus comprising means to implement of any of Examples 1-21.

Example 24 is a system to implement of any of Examples 1-21.

Example 25 is a method to implement of any of Examples 1-21.

In some aspects, the Examples described can include any one or combination of the apparatus and system examples above including any one or combination of the individual features disclosed herein.

### BRIEF DESCRIPTION OF THE FIGURES

The above-mentioned and other features and advantages of this disclosure, and the manner of attaining them, will become more apparent and the disclosure itself will be better understood by reference to the following description of examples taken in conjunction with the accompanying drawings, wherein:

FIG. 1 is an anatomic view of a shoulder joint of a patient.

FIG. 2 is a schematic diagram of a prosthesis assembly implanted in a humerus of the patient and forming at least a portion of the shoulder joint.

FIG. 3 is an exploded view of a humeral trial system according to an example of the present application.

FIG. 4 is a perspective view of the humeral trial system of FIG. 3 as an assembly.

FIG. 4A is another perspective view of the assembly of FIG. 4.

FIG. 5A is a plan view of a trial bearing of the humeral trial system of FIGS. 3 and 4.

FIG. 5B is a perspective view of the trial bearing of FIG. 5A.

FIGS. 6A and 6B are perspective views of a humeral trial tray of the humeral trial system of FIGS. 3 and 4.

FIG. 6C is a plan view of the humeral trial tray of FIGS. 6A and 6B.

FIGS. 7A and 7B are perspective views of a connector and a post of the humeral trial system of FIGS. 3 and 4.

FIG. 8 is a perspective view showing assembly of the connector with the humeral trial tray according to an example of the present application.

FIGS. 9A-9C are cross-sectional views showing adjustment of the trial bearing with respect to the trial tray according to an example of the present application.

Corresponding reference characters indicate corresponding parts throughout the several views. The exemplifications set out herein illustrate examples of the disclosure, and such exemplifications are not to be construed as limiting the scope of the disclosure any manner.

### DETAILED DESCRIPTION

In describing the examples of the disclosure illustrated and to be described with respect to the drawings, specific terminology will be used for the sake of clarity. However, the disclosure is not intended to be limited to any specific terms or illustrations used herein, and it is to be understood that each specific term includes all technical equivalents.

The present disclosure is directed to orthopedic trial apparatuses and orthopedic systems for trialing that can be used in joint replacement procedures such as a reverse shoulder arthroplasty. Although the present apparatuses and systems are being described in reference to a reverse shoulder arthroplasty, the apparatuses and systems can be used for other procedures and other joints as discussed previously.

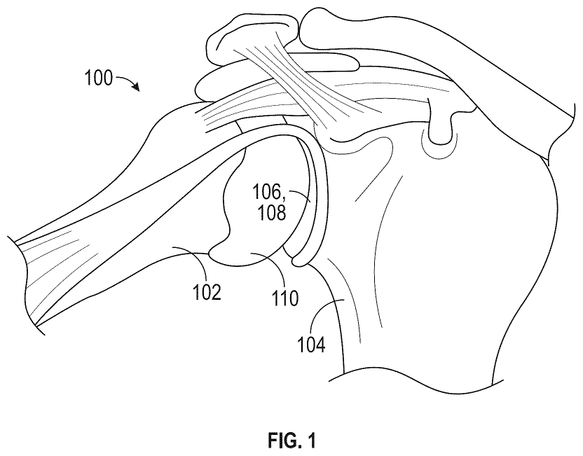

FIG. 1 illustrates a shoulder joint 100 with several ligaments stripped away. As shown, the shoulder joint 100 includes a humerus 102 and a scapula 104 that has a glenoid 106 with a socket 108 for interacting with a humeral head 110 of humerus 102. Humeral head 110 can articulate within a socket 108 to allow for normal motion of the shoulder joint 100. Disease can degenerate the bone or soft tissue of the humeral head 110 and/or scapula 104. These can cause pain and/or can negatively impact shoulder joint function. Typically, a surgical intervention may be required to replace the shoulder joint and restore shoulder joint function.

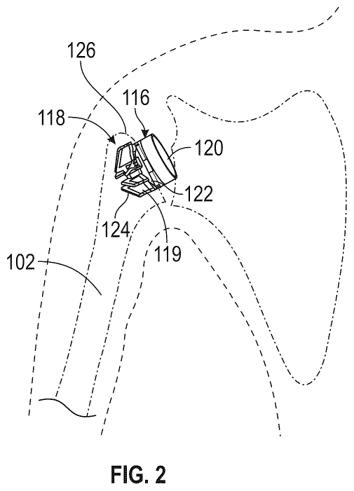

FIG. 2 illustrates a schematic diagram of a prosthesis assembly 116 installed at the shoulder joint 100. For simplicity, a corresponding prosthesis assembly installed in the scapula is not illustrated. The prosthesis assembly 116 can include a humeral implant 118 and a bearing 120. The humeral implant 118 can include a humeral tray 122 and a stem and/or stemless anchor 124.

The humeral implant 118 can be fitted into a recess 119 formed at a proximal end portion 126 of the humerus 102. The embodiment of FIG. 2 show is a fin design for the stem and/or stemless anchor 124 that has a reduced length in a longitudinal direction. However, a more traditional stem design is also contemplated for the stem and/or stemless anchor 124. As shown in FIG. 2, the humeral tray 122 can interface with and can be coupled to the bearing 120 and the stem and/or stemless anchor 124. The bearing 120 can couple with the humeral tray 122 using locking mechanisms. The stem and/or stemless anchor 124 can be configured in the manner of the Sidus® Stem-Free Shoulder prosthesis, Comprehensive® Nano Stemless Shoulder or another commercially available and manufactured by Zimmer Biomet Inc., of Warsaw Indiana. The stemless anchor 124 design, the humeral tray 122 design and the bearing 120 design are purely exemplary and are provided merely to facilitate practitioner understanding of implants that can be trialed using the trialing systems and components disclosed herein.

Reverse total shoulder arthroplasty is one of several types of shoulder replacement surgeries. In reverse total shoulder arthroplasty, a portion of a patient's humerus and a portion of the patient's glenoid is replaced and/or augmented with implantable components. With a reverse shoulder arthroplasty, prosthesis components include a glenoid implant (not specifically shown in FIG. 2) that acts as the “ball” (semi-spherical or otherwise shaped to replicate the head of the humerus) and the bearing 120 shaped as a cup to receive the “ball”. The bearing 120 acts as the glenoid, in reverse shoulder arthroplasty, with the “ball” on the glenoid side of the shoulder joint. In other words, the relationship between the prosthesis components in the surgically created shoulder joint is opposite that of the anatomically correct shoulder joint.

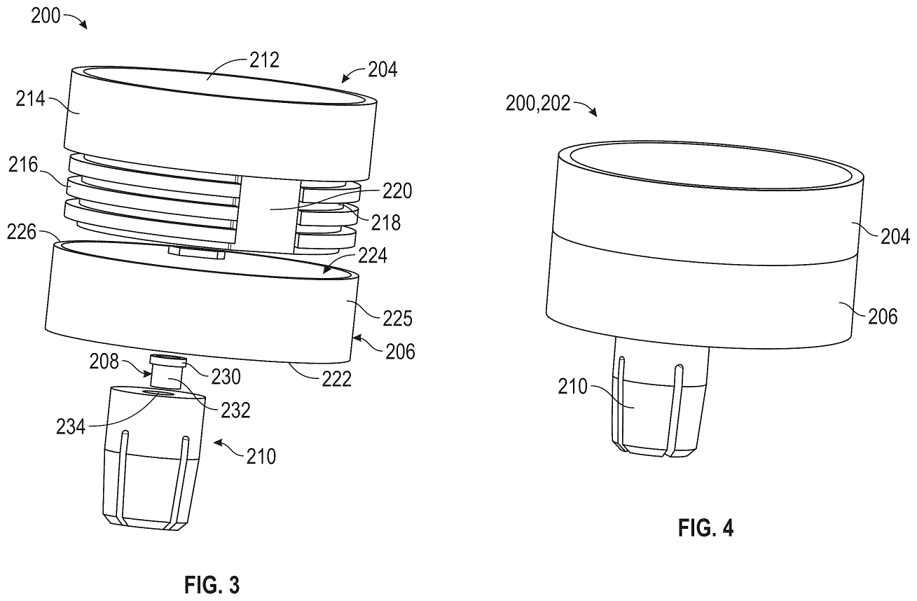

FIG. 3 shows an example of a humeral trial system 200 configured to simulate the size, orientation and/or shape of the bearing 120 and/or the humeral tray 122 of FIG. 2. FIG. 4 shows the humeral trial system 200 coupled together as a humeral trial assembly 202. As shown in the exploded view of FIG. 3, the humeral trial system 200 can include a trial bearing 204, a trial tray 206, a connector 208 and a post 210. FIG. 4 illustrates the trial bearing 204, trial tray 206 and the post 210.

The humeral trial system 200 and the humeral trial assembly 202 can be temporary components that when coupled together simulate assembly of the humeral tray 122 and bearing 120 of FIG. 2. The humeral trial system 200 and the humeral trial assembly 202 can be coupled to the stemless anchor 124 of FIG. 2, for example, or another component such as a trial implant. The humeral trial system 200 and the humeral trial assembly 202 can be used for sizing the bearing 120 (previously shown in FIG. 2) and/or to perform joint kinematics such as range of motion.

As shown in FIG. 3, the trial bearing 204 can include an articular surface 212, an outer edge 214 or side, a plurality of tabs 216, a plurality of grooves 218 (e.g., reliefs) and at least one window 220. The trial bearing 204 can be formed of a polymeric material, for example. The polymeric material can be silicone, polyphenylsulfone (PPSU), polyetheretherketone (PEEK), High Density Poly Ethylene (HDPE) or other suitable biocompatible polymeric material, for example. The trial bearing 204 can be available in a range of sizes that replicate/simulate standard sizes for the bearing 120 (FIG. 2) including designs with different angulation and diameters. The articular surface 212 can form a proximal most side of the trial bearing 204 and can be cup or bowl shape to receive the ball component installed on the glenoid. The articular surface 212 can be angled or tilted according to some examples, which are not specifically shown. The articular surface 212 can extend to adjacent the outer edge 214. Outer edge 214 can form the side of the trial bearing 204 and can include the features such as the plurality of tabs 216, the plurality of grooves 218 (e.g., reliefs) and the at least one window 220. The outer edge 214 can extend generally distally from at or adjacent the articular surface 212 to a distal side 222. The plurality of tabs 216, the plurality of grooves 218 and the at least one window 220 can form part of the outer edge 214.

The plurality of tabs 216 can be shaped projections configured to engage female features such as recesses (not shown) of the trial tray 206 as further described and shown herein. Similarly, the plurality of grooves 218 can be configured (shaped, sized, etc.) to receive corresponding male features (not shown) of the trial tray 206. The plurality of tabs 216 can be circumferentially arranged along the side of the trial bearing 204 forming part of the side thereof. The plurality of tabs 216 can be spaced from one another by respective ones of the plurality of grooves 218. Such spacing can be uniform, for example.

The plurality of grooves 218 can comprise gaps or reliefs between adjacent of the plurality of tabs 216. The plurality of grooves 218 and the plurality of tabs 216 can extend around less than an entirety of a circumference of the outer edge 214 of the trial bearing 204. The plurality of grooves 218 and the plurality of tabs 216 can be interrupted by the at least one window 220. The at least one window 220 can be a groove and/or a featureless part of the outer edge 214, for example. Thus, the at least one window 220 can be devoid of the plurality of tabs 216 and the plurality of grooves 218. The present example contemplates two windows for the at least one window 220 on opposing sides of the outer edge 214. However, other examples contemplate use of a single window or three or more windows spaced at intervals around the circumference.

Still referring to FIG. 3, the trial tray 206 can be a cylindrical shell or can be otherwise shaped including a recess or cavity 224 defined by a wall 225 with an opening at a proximal side 226 configured to receive part of the trial bearing 204 within the recess or cavity 224. The trial tray 206 can include additional features which will be discussed in further detail subsequently. The trial tray 206 can be formed of plastic, metal, composite, ceramic or other suitable material. The trial tray 206 can include a distal side 228 that opposes the proximal side 226. The distal side can be generally flat or otherwise shaped as desired (e.g., bowl shaped, tapered shape, etc.). Similarly, although shown a generally cylindrical in FIG. 3, the trial tray 206, in particular the wall 225 can be otherwise shaped (e.g., bowl shaped, tapered shape, etc.) according to further embodiments.

The connector 208 can extend at least partially from the distal side 228 of the trial tray 206 and can be captured at least partially in the cavity 224 of the trial tray 206. The connector 208 can be a fastener or other component. The connector 208 can include a head portion 230 having an enlarged diameter relative to a body portion 232. The body portion 232 can be threaded or otherwise configured (e.g., tapered for press-fit) to couple with the post 210 via an aperture 234, for example. The post 210 can have an elongate length and can be configured to be received by the stem and/or stemless anchor 124 (FIG. 2) or a trial implant (not shown) for coupling the humeral trial system 200 when assembled to the stem and/or stemless anchor 124 (FIG. 2) or the trial implant (not shown). An exemplary configuration for the post 210 is shown in FIG. 3. Although not specifically illustrated, various different stem designs having different shapes (e.g., oval in cross-section) and lengths can be included as part of the humeral trial system 200. The design for the post 210 selected can depend upon the implant system being utilized by the physician.

FIGS. 4 and 4A show the humeral trial assembly 202 created from the humeral trial system 200. As shown in FIGS. 4 and 4A, the trial bearing 204 is mounted atop and received at least partially within the trial tray 206. As shown in FIG. 4, the post 210 is coupled to the trial tray 206 such as via the connector 208 (FIGS. 3 and 4A).

FIG. 4A shows the articular surface 212 of the trial bearing 204. The trial bearing 204 such at the articular surface 212 can include an aperture 236 such a slot 238 that can provide access to the connector 208 for a tool to allow for selective movement of the connector 208 and the post 210 (FIG. 4) relative to the trial tray 206 along a slot (not shown) as further discussed herein. In particular, via the aperture 236, the tool (not shown) can be inserted through the trial bearing 204 and into the trial tray 206 with the components mounted together. The tool (not shown) can be shaped to engage a coupling feature of the head portion 230 of the connector 208 for loosening and facilitating movement of the connector 208.

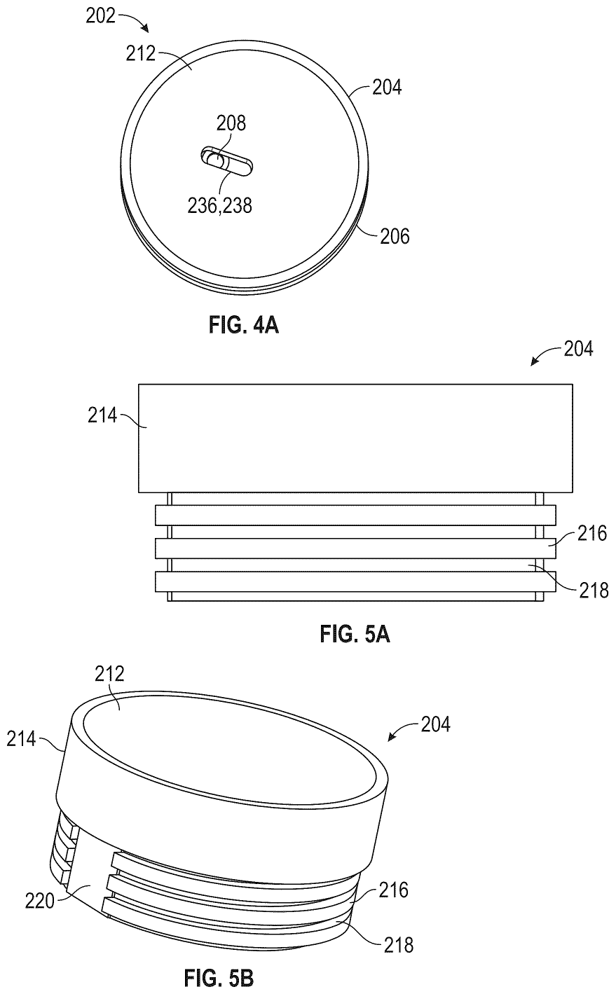

FIGS. 5A and 5B show the trial bearing 204 from various perspectives. FIGS. 5A and 5B show the outer edge 214 or side, the plurality of tabs 216 and the plurality of grooves 218 (e.g., reliefs). FIG. 5B additionally shows the articular surface 212 and the at least one window 220 as previously discussed.

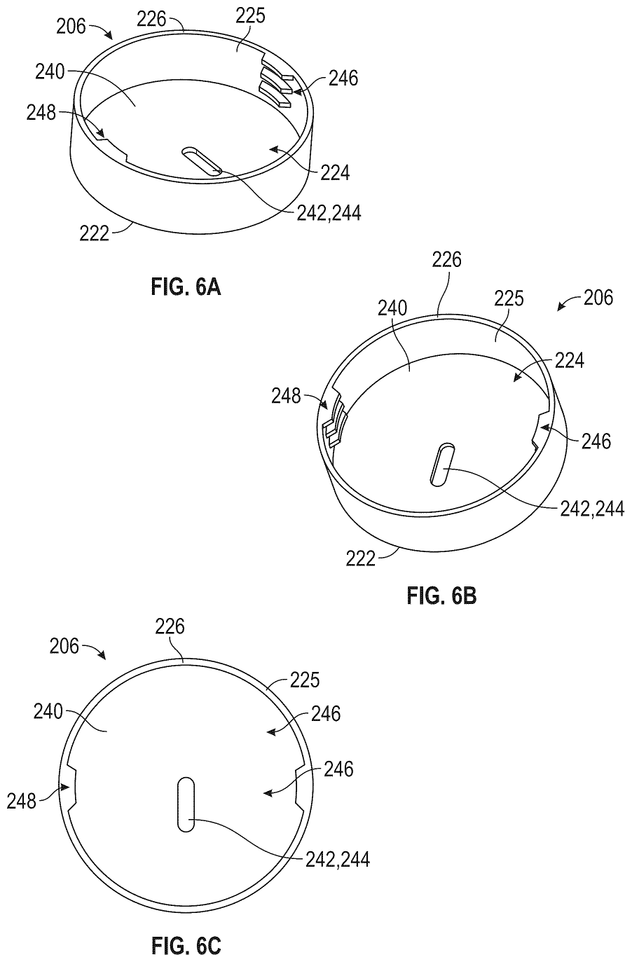

FIGS. 6A-6C show the trial tray 206 from various perspectives. The trial tray 206 can include the recess or cavity 224, the wall 225, the proximal side 226 and the distal side 228 (FIGS. 6A and 6B). FIGS. 6A-6C additionally illustrate a distal surface 240 having an aperture 242 such as a slot 244. The wall 225 can include a first plurality of tabs 246 and a second plurality of tabs 248.

The distal surface 240 can comprise a bottom of the cavity 224 and can be connected with the wall 225. The aperture 242 can extend through the distal surface 240 and is configured to receive at least a portion of the connector 208 (FIGS. 3 and 4A). The aperture 242 is shaped as the slot 244 to allow for adjustment of the offset of the post 210 (FIGS. 3 and 4) relative to the trial tray 206 via the connector 208 (FIGS. 3 and 4A).

The first plurality of tabs 246 and the second plurality of tabs 248 are connected to the wall 225 and extend generally radially inward therefrom toward a centerline of the trial tray 206. As shown in FIG. 6A, the first plurality of tabs 246 comprise spaced projections, for example. The first plurality of tabs 246 can be spaced at a uniform distance and can be shaped to be received by the plurality of grooves 218 (FIGS. 3, 5A and 5B). When received by the plurality of grooves 218, the first plurality of tabs 246 can engage the plurality of tabs 216 (FIGS. 3, 5A and 5B).

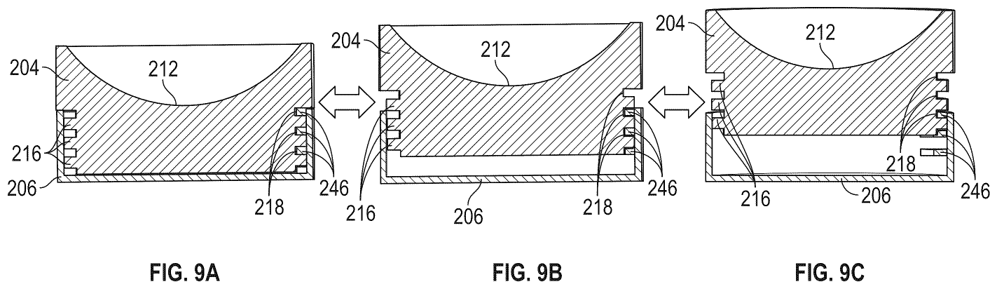

The second plurality of tabs 248 can be arranged on an opposing side of the wall 225 from the first plurality of tabs 246. The second plurality of tabs 248 comprise spaced projections, for example having a similar shape and geometric height spacing as the first plurality of tabs 246 form the centerline axis. The second plurality of tabs 248 can be spaced at a uniform distance and can be shaped to be received by the plurality of grooves 218 (FIGS. 3, 5A and 5B). When received by the plurality of grooves 218 (FIGS. 3, 5A and 5B), the second plurality of tabs 248 can engage the plurality of tabs 216 (FIGS. 3, 5A and 5B). Examples of this arrangement are shown in FIGS. 9A-9C discussed subsequently.

The first plurality of tabs 246 or the second plurality of tabs 248 are sized to be received by the at least one window 220 (FIGS. 3 and 5B) when aligned therewith. Such alignment of the first plurality of tabs 246 or the second plurality of tabs 248 with the at least one window 220 (FIGS. 3 and 5B) can facilitate insertion and proximal adjustment of the trial bearing 204 relative to the trial tray 206 as further described in reference to FIGS. 9A-9C.

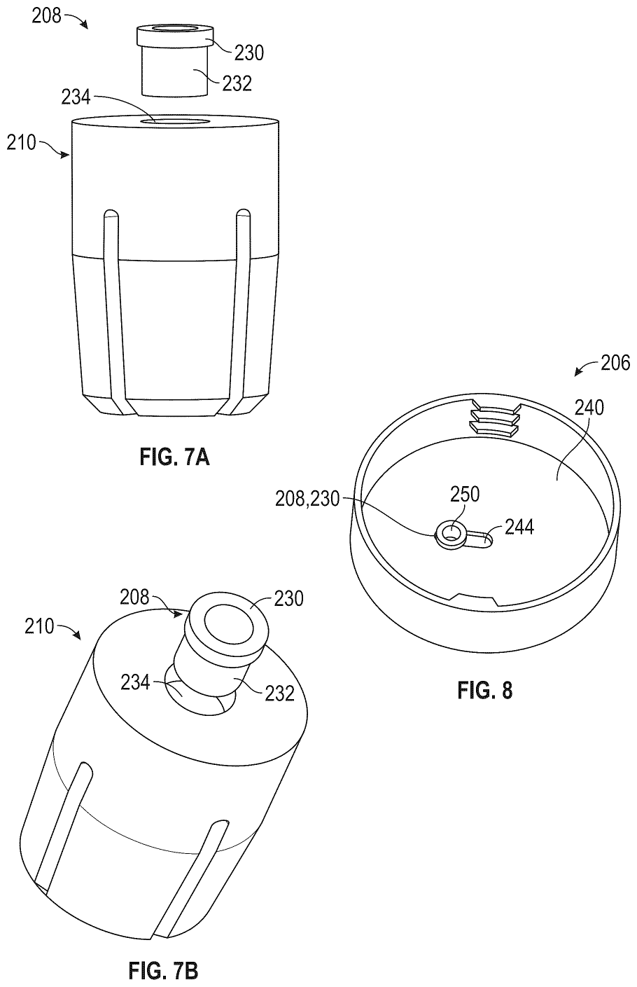

FIGS. 7A and 7B show the connector 208 and the post 210 exploded from one another and further illustrates the head portion 230, the body portion 232 and the aperture 234. FIG. 8 shows the connector 208 received by the slot 244 of the trial tray 206 with the distal surface 240 engaged by the head portion 230. The slot 244 is sized such that the body portion 232 (FIGS. 7A and 7B) can pass through the slot 244 but the head portion 230 having a larger diameter cannot pass through the slot 244.

As shown in FIG. 8, the head portion 230 can have a coupling feature 250 such as a polygonal recess or the like that can be engaged by the tool as discussed in regard to FIG. 4A. The tool can be used to engage the coupling feature 250 of the head portion 230 of the connector 208 for loosening engagement between the head portion 230 and the distal surface 240. Once loosened, the connector 208 (and the post 210 although now shown) can be moved along the slot 244 relative to the trial tray 206 to a desired position. Once such desired position is achieved, the tool can be used to tighten the connector 208 back into engagement between the head portion 230 and the distal surface 240.

FIGS. 9A-9C are cross-sections of the trial tray 206 and the trial bearing 204 and illustrate a method of adjusting a height of the articular surface 212 of the trial bearing 204 relative to the trial tray 206. FIG. 9A shows the trial bearing 204 fully seated down into the trial tray 206 in a first position with the trial bearing 204 positioned relative to the trial tray 206 such that the first plurality of tabs 246 are received by the plurality of grooves 218. The second plurality of tabs 248 (FIGS. 6A-6C) are not illustrated in FIGS. 9A-9C due to the cross-section selected. In the first position, the first plurality of tabs 246 interface with and engage the plurality of tabs 216 on proximal and distal sides thereof. If the height of the articular surface 212 of the trial bearing 204 is desired to be adjusted higher relative to the trial tray 206 from the first position of FIG. 9A, the trial bearing 204 is rotated relative to the trial tray 206 to align the first plurality of tabs 246 with the at least one window 220 (FIGS. 3 and 5B). Then the trial bearing 204 can be moved with a generally linear proximal movement (proximal lifting) of the trial bearing 204 relative to the trial tray 206 to another position. Once a desired height for the articular surface 212 of the trial bearing 204 relative to the trial tray 206 is achieved, the trial bearing 204 is coupled back with the trial tray 206. This occurs by rotating/clocking the trial bearing 204 relative to the trial tray 206 to bring the first plurality of tabs 246 out of alignment with the at least one window 220 (FIGS. 3 and 5B) and having the first plurality of tabs 246 again be received by the plurality of grooves 218.

FIG. 9B shows the trial bearing 204 re-adjusted to a second position (a second height) relative to the trial tray 206 following the process described above. The height for the articular surface 212 of the trial bearing 204 in FIG. 9B is higher than that of FIG. 9A. In the second position, only some of the first plurality of tabs 246 are received by the plurality of grooves 218 as some of the plurality of grooves 218 and some of the plurality of tabs 216 are positioned proximal of a proximal rim (a proximal side) of the trial tray 206. Put another way, at least one of the first plurality of tabs 246 is located distal of the plurality of grooves 218 in FIG. 9B.

FIG. 9C shows the trial bearing 204 readjusted to a third position (a third height) relative to the trial tray 206. The height for the articular surface 212 of the trial bearing 204 in FIG. 9C is higher than that of FIG. 9A and FIG. 9B. In the third position, only one of the first plurality of tabs 246 are received by one of the plurality of grooves 218. At least two of the plurality of grooves 218 and at least two of the plurality of tabs 216 are positioned proximal of a proximal rim (a proximal side) of the trial tray 206. Put another way, two of the first plurality of tabs 246 are located distal of the plurality of grooves 218 in FIG. 9C.

Thus, FIGS. 9A-9C and other FIGURES here depict a method of trialing for an arthroplasty including: providing a trial tray and a trial bearing, aligning the trial bearing with the trial tray in a first orientation, inserting the trial bearing down onto the trial tray in the first orientation and rotating the trial bearing relative to the trial tray from the first orientation to a second orientation to couple the trial bearing to the trial tray. The method includes rotating the trial bearing relative to the trial tray from the second orientation back to the first orientation. In the first orientation, the method includes linearly translating the trial bearing relative to the trial tray to adjust a height of an articular surface of the trial bearing relative to the trial tray (e.g., moving from first position of FIG. 9A to another position of FIG. 9B or 9C). As described above with regard to FIG. 9A and with reference to FIG. 3 and FIG. 5B, aligning the trial bearing with the trial tray includes aligning a plurality of tabs of the trial tray with at least one window of the trial bearing, and wherein rotating the trial bearing relative to the trial tray from the first orientation to a second orientation inserts one or more of the plurality of tabs into one or more of a plurality of grooves of the trial bearing. As described for example with regard to FIGS. 3 and 8, the method can include connecting a post to the trial tray using a connector inserted in a slot of the trial tray and adjusting a position of the post relative to the trial tray by moving the connector along the slot. As illustrated in FIG. 4A, the method can include accessing the connector for movement in the slot of the trial tray via an aperture in trial bearing.

The term “proximal” refers to the general orientation of the side and/or surface when the humeral implant is implanted in the bone. Thus, “proximal” refers to a direction or location generally in the direction of or toward the head of a patient, and “distal” refers to the opposite direction of proximal, i.e., away from the head of a patient. As used herein, the terms “anterior” and “posterior” should be given their generally understood anatomical interpretation. Thus, “posterior” refers to a location or direction generally toward a rear of the patient. Similarly, “anterior” refers to a location or direction generally toward a front of the patient. Thus, “posterior” refers to the opposite direction of “anterior.” Similarly, the terms “medial” and “lateral” should be given their generally understood anatomical interpretation. “Medial” refers to the more inward facing (inner part) of the prosthesis or guide (when in the implanted orientation) and “lateral” refers to the outer part or outward facing part. “Medial” refers to the opposite direction of “lateral.” The terms “substantially”, “generally” or “about” means within 10% of the value discussed.

It will be readily understood to those skilled in the art that various other changes in the details, material, and arrangements of the parts and method stages which have been described and illustrated in order to explain the nature of the inventive subject matter can be made without departing from the principles and scope of the inventive subject matter as expressed in the subjoined claims. For example, the order of method steps or stages can be altered from that described above, as would be appreciated by a person of skill in the art.

It will also be appreciated that the various dependent claims, examples, and the features set forth therein can be combined in different ways than presented above and/or in the initial claims. For instance, any feature(s) from the above examples can be shared with others of the described examples, and/or a feature(s) from a particular dependent claim may be shared with another dependent or independent claim, in combinations that would be understood by a person of skill in the art.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A modular trial assembly for an arthroplasty comprising:
a trial tray having an aperture therein;
a trial bearing coupled to the trial tray and configured to be selectively moveable relative thereto to adjust a height of an articular surface of the trial bearing relative to the trial tray;
a connector received by the aperture and extending from the trial tray; and
a post coupled to the connector and positioned adjacent the trial tray, wherein the post is configured to be received by an orthopedic prosthesis for coupling the modular trial assembly to the orthopedic prosthesis.

2. The modular trial assembly of claim 1, wherein the aperture is a slot and the connector is selectively moveable along the slot to adjust a position of the post relative to the trial tray.

3. The modular trial assembly of claim 2, wherein the trial bearing includes a slot therein, wherein the slot of the trial bearing allows for access to the connector and the slot of the trial tray.

4. The modular trial assembly of claim 1, wherein the trial tray includes a plurality of tabs, wherein the plurality of tabs are spaced apart at a desired distance, and wherein one or more of the plurality of tabs are configured to be received in one or more of a plurality of grooves of the trial bearing.

5. The modular trial assembly of claim 4, wherein the trial tray and the trial bearing are selectively coupled via the one or more of the plurality of tabs being received by the one or more of the plurality of grooves, and wherein the trial bearing is selectively rotatable relative to the trial tray to remove the one or more of the plurality of tabs from the one or more of the plurality of grooves to allow for selective movement of the trial bearing relative to the trial tray to adjust the height of the articular surface of the trial bearing.

6. The modular trial assembly of claim 5, wherein the trial bearing is selectively rotatable to align the plurality of tabs with at least one window of the trial bearing to allow for selective movement of the trial bearing relative to the trial tray to adjust the height of the articular surface of the trial bearing.

7. An orthopedic system for an arthroplasty comprising:
a trial tray;
a trial bearing configured to be selectively coupled to and decoupled from the trial tray by rotation of the trial bearing relative to the trial tray, wherein, when decoupled from the trial tray, the trial bearing is selectively moveable to adjust a height of an articular surface of the trial bearing relative to the trial tray;
a post configured to be received by an orthopedic prosthesis; and
a connector configured to couple the post with the trial tray.

8. The orthopedic system of claim 7, wherein, when the connector is coupled to the trial tray and the post, the trial tray includes a slot and the connector is configured to be selectively movable along the slot adjust a position of the post relative to the trial tray.

9. The orthopedic system of claim 8, the trial bearing includes a slot therein, wherein, when the trial bearing is coupled to the trial tray, the slot of the trial bearing allows for access to the connector and the slot of the trial tray.

10. The orthopedic system of claim 7, wherein the trial tray includes a plurality of tabs, wherein the plurality of tabs are spaced apart at a desired distance, and wherein when the trial bearing and the trial tray are coupled, one or more of the plurality of tabs are configured to be received in one or more of a plurality of grooves of the trial bearing, and wherein the trial bearing is selectively rotatable relative to the trial tray to remove the one or more of the plurality of tabs from the one or more of the plurality of grooves to allow for selective movement of the trial bearing relative to the trial tray to adjust the height of the articular surface of the trial bearing.

11. The orthopedic system of claim 10, wherein the trial bearing is selectively rotatable to align the plurality of tabs with at least one window of the trial bearing to allow for selective movement of the trial bearing relative to the trial tray to adjust the height of the articular surface of the trial bearing.

12. A modular trial assembly for an arthroplasty comprising:
a trial tray having an aperture therein;
a trial bearing coupled to the trial tray and configured to be rotatable and selectively linearly translatable relative to the trial tray to adjust a height of an articular surface of the trial bearing relative to the trial tray; and
a post coupled to the trial tray and connecting the modular trial assembly to an orthopedic prosthesis.

13. The modular trial assembly of claim 12, further comprising a connector connecting the post to the trial tray, wherein the connector is received in a slot of the trial tray, and wherein the connector is selectively moveable along the slot to adjust a position of the post relative to the trial tray.

14. The modular trial assembly of claim 13, wherein the trial bearing includes a slot therein, wherein the slot of the trial bearing allows for access to the connector and the slot of the trial tray.

15. The modular trial assembly of claim 12, wherein the trial tray includes a plurality of tabs, wherein the plurality of tabs are spaced apart at a desired distance, and wherein one or more of the plurality of tabs are configured to be received in one or more of a plurality of grooves of the trial bearing, wherein the trial tray and the trial bearing are selectively coupled via the one or more of the plurality of tabs being received by the one or more of the plurality of grooves, and wherein the trial bearing is selectively rotatable relative to the trial tray to remove the one or more of the plurality of tabs from the one or more of the plurality of grooves to allow for selective movement of the trial bearing relative to the trial tray to adjust the height of the articular surface of the trial bearing.

16. The modular trial assembly of claim 15, wherein the trial bearing is selectively rotatable to align the plurality of tabs with at least one window of the trial bearing to allow for selective movement of the trial bearing relative to the trial tray to adjust the height of the articular surface of the trial bearing.

17. A method of trialing for an arthroplasty comprising:
providing a trial tray and a trial bearing;
aligning the trial bearing with the trial tray in a first orientation;
inserting the trial bearing down onto the trial tray in the first orientation; and
rotating the trial bearing relative to the trial tray from the first orientation to a second orientation to couple the trial bearing to the trial tray.

18. The method of claim 17, further comprising:
rotating the trial bearing relative to the trial tray from the second orientation back to the first orientation; and
in the first orientation, linearly translating the trial bearing relative to the trial tray to adjust a height of an articular surface of the trial bearing relative to the trial tray.

19. The method of claim 17, wherein aligning the trial bearing with the trial tray includes aligning a plurality of tabs of the trial tray with at least one window of the trial bearing, and wherein rotating the trial bearing relative to the trial tray from the first orientation to a second orientation inserts one or more of the plurality of tabs into one or more of a plurality of grooves of the trial bearing.

20. The method of claim 17, further comprising:
connecting a post to the trial tray using a connector inserted in a slot of the trial tray;
adjusting a position of the post relative to the trial tray by moving the connector along the slot; and
accessing the connector for movement in the slot of the trial tray via an aperture in trial bearing.
